(19) United States
(12) Patent Application Publication
(10) Pub. No.: US 2026/0036585 A1
(43) Pub. Date: Feb. 5, 2026

(54) SYSTEMS AND METHODS FOR MULTIPLEXED DETECTION OF PROTEINS IN COMPLEX FLUIDS

(71) Applicant: GUARDANT HEALTH, INC., Palo Alto, CA (US)

(72) Inventors: James GHADIALI, San Diego, CA (US); Andrew KENNEDY, San Diego, CA (US)

(21) Appl. No.: 19/354,723

(22) Filed: Oct. 9, 2025

Related U.S. Application Data
(63) Continuation of application No. PCT/US2024/024153, filed on Apr. 11, 2024.
(60) Provisional application No. 63/495,432, filed on Apr. 11, 2023.

(51) Int. Cl. G01N 33/574; C12Q 1/6806; C12Q 1/6851; G01N 33/543

(52) U.S. Cl. CPC G01N 33/574; C12Q 1/6806; C12Q 1/6851; G01N 33/54326; C12Q 2600/16; G01N 2446/00; G01N 2458/10; G01N 2470/04

(57) ABSTRACT

Multiplex analysis methods and systems for accurately detecting and quantitating multiple analytes in a sample are disclosed. The sample is contacted with an analyte capturing agent and immobilized on a magnetic particle, followed by magnetic separation and washing of the particle and bound analyte. This complex is then contacted with a detection agent labeled with an oligonucleotide barcode specific to the analyte target, followed by quantitative measurement of the barcode by qPCR or NGS. The combination of a plurality of analyte capture particles and cognate detection probes allows multiple analytes to be assayed simultaneously and in a multiplex manner.

### CROSS-REFERENCE TO RELATED APPLICATIONS

This application is a Continuation of PCT Application No. PCT/US2024/024153, filed Apr. 11, 2024, which claims the benefit of priority of U.S. Provisional Patent Application No. 63/495,432, filed Apr. 11, 2023, which is incorporated by reference herein in its entirety for all purposes.

### TECHNICAL FIELD

The present disclosure relates to systems and methods for accurately detecting and measuring one or more analytes in a sample in a multiplexed reaction system, for example a multiplexed immuno-PCR assay system.

### BACKGROUND

The enzyme linked immunosorbent assay (ELISA) is a well-known immunoassay that uses affinity reagent pairs to capture and label a target molecule with signal amplification such that the concentration of the target molecule can be determined. ELISA assays have certain limitations such as tedious and laborious assay procedures and insufficient level of specificity or sensitivity. Also, standard ELISA assays are not readily amenable to multiplexing analytes within a single well.

Certain methods, such as CiteSeq (Cellular Indexing of Transcriptomes and Epitopes by Sequencing) or AbSeq, employ oligonucleotide tagged antibodies for detection of proteins on the surface of cells, but are not extended to soluble protein detection in complex milieu. Other methods, such as immuno-PCR, have not been used in multiplex detection methods due to the complexity of multiple capture antibodies being employed in a single well.

The Immuno-PCR (iPCR) assay combines the versatility of immunoassays with the exponential signal amplification power of polymerase chain reaction (PCR). Typically, iPCR allows a 10-1000-fold increase in sensitivity over an analogous ELISA. However, a need exists for a multiplex analysis method and system for detecting and/or quantitating multiple analytes in a sample. Such a multiplex analysis method should be precise and accurately measure multiple analytes at concentrations over several orders of magnitude.

### SUMMARY

The present disclosure is directed to systems and methods for high sensitivity multiplexed analyte detection and/or quantitation in complex biofluids that operates over a broad dynamic range of analyte concentration. In certain aspects, the systems and methods relate to multiplexed protein biomarker detection by means of iPCR combined with quantitative Polymerase Chain Rection (qPCR) or Next Generation Sequencing (NGS) readout, further enabled with magnetic protein separation from complex milieu.

According to various embodiments of the method, initial analyte capture from a sample is achieved using a capture agent bound or linked to a magnetic particle. A set of magnetic particles is provided, each magnetic particle in the set having a capture agent operatively linked to the particle. Each capture agent is configured to specifically bind to a distinct analyte. The set of magnetic particles is combined with the sample and each capture agent is allowed to specifically bind to the analyte, producing a complex containing the magnetic particle, capture agent and analyte. The formed capture agent-analyte complex linked to the magnetic particle is separated from the sample milieu by placing in a magnetic field, allowing for separation and washing of the complex from potentially interfering compounds.

According to other embodiments of the method, initial analyte capture from a sample is achieved using a capture agent that is not bound or linked to a magnetic particle, or prior to binding or linking and immobilization of the capture agent to the magnetic particles. A set of capture agents is provided, each capture agent configured to specifically bind to a distinct analyte. The set of capture agents is combined with the sample in a solution and each capture agent is allowed to specifically bind to the analyte, forming a capture agent-analyte complex. The capture agent with bound analyte is subsequently operatively linked to magnetic particles to form a capture agent-analyte complex linked to magnetic particles. The formed capture agent-analyte complex linked to the magnetic particle is separated from the sample milieu by placing in a magnetic field, allowing for separation and washing of the complex from potentially interfering compounds.

According to additional embodiments of the method, initial analyte capture from a sample is achieved using a substrate having a plurality of capture agents operatively-linked thereto. In some embodiments, the substrate is one or more well of a plastic microplate. The substrate is provided having a plurality of capture agents operatively-linked to the substrate, each capture agent configured to specifically bind to a distinct analyte. The sample is combined with the substrate to allow each capture agent to specifically bind to the analyte, to form a capture agent-analyte complex on the substrate. The substrate and capture agent-analyte complex is washed to remove unbound compounds and potentially interfering compounds.

According to various embodiments, a set of detection agents is provided, and each detection agent in the set is configured to specifically bind to the capture agent-analyte complex linked to the magnetic particle, or the capture agent-analyte complex on the substrate. In addition, each detection agent has a specific oligonucleotide barcode sequence operatively linked to the detection agent.

In some embodiments, the washed capture agent-analyte complex linked to the magnetic particle is combined with the set of detection agents, and each detection agent is allowed to specifically bind to the capture agent-analyte complex to produce a capture agent-analyte-detection agent complex linked to the magnetic particle. This complex is separated from the mixture and excess reagent is removed by magnetic separation and washing.

In other embodiments, the washed capture agent-analyte complex on the substrate is combined with the set of detection agents, and each detection agent is allowed to specifically bind to the capture agent-analyte complex to produce a capture agent-analyte-detection agent complex on the substrate. The substrate and capture agent-analyte-detection agent is washed to remove excess reagent.

According to various embodiments, a quantitative measuring step is performed on the washed capture agent-analyte-detection agent complex, specifically on the oligonucleotide barcode sequence, to detect and/or quantitate each of the analytes. Embodiments of the method include performing multiplex quantitative PCR (qPCR) to detect and/or quantitate the analytes. Other embodiments include performing next generation sequencing (NGS) to detect and/or quantitate the analytes.

According to various embodiments of the disclosure, the abundance of the barcode oligonucleotide in the capture agent-analyte-detection agent complex provides a proxy for the abundance of the analyte. In some embodiments, the amount of barcode oligonucleotide is quantified through qPCR using an oligonucleotide-specific fluorescent hydrolysis probe (e.g., TaqMan®) in a multiplexed assay. In some embodiments, the qPCR is preceded by one or more rounds of PCR amplification of the oligonucleotide.

In other embodiments, the amount of barcode oligonucleotide is quantified through NGS. According to various embodiments, the multiplexing capacity of the assay method is substantially expanded by a NGS readout of the oligonucleotide abundance. In some embodiments, the NGS readout is preceded by one or more rounds of PCR amplification of the oligonucleotide.

According to various embodiments, by utilizing a plurality of specific barcodes associated with a plurality of cognate capture agent/detection agent pairs, multiple analytes are detected within a single assay. This multiplexing analysis vastly exceeds that of standard quantitative PCR which may be limited to 4-5 analytes per reaction. In some embodiments of the present methods and systems, at least six analytes, or forty or more analytes, are detected and/or quantitated in a single multiplex analysis. In some embodiments, multiple barcode-specific Taqman probes are used in a qPCR assay. In some embodiments, any number of analyte-specific barcodes are used with a NGS readout.

In some embodiments, qPCR would be an appropriate readout if only 1-4 or 5 analytes are required. This would enable faster turnaround and may be more cost-effective than NGS. In some embodiments, higher levels of multiplexing would necessitate NGS due to inherent limitations of qPCR. In some embodiments, when multiple proteins are being analyzed (>5), 1-5 proteins are assayed rapidly by qPCR, and the additional proteins are quantified by NGS.

In some embodiments, the present disclosure provides a method for detecting and/or quantitating one or more analytes in a sample, the method comprising: providing a set of magnetic particles, each magnetic particle in the set comprising a capture agent operatively-linked to the particle, each capture agent configured to specifically bind to a distinct analyte; combining the sample and set of magnetic particles to allow each capture agent to specifically bind to the analyte, to produce a capture agent-analyte complex linked to the magnetic particle; separating and washing the capture agent-analyte complex; providing a set of detection agents, each detection agent in the set configured to specifically bind to the capture agent-analyte complex, and each detection agent comprising a specific oligonucleotide barcode sequence operatively linked to the detection agent; combining the washed capture agent-analyte complex and the set of detection agents to allow the detection agent to specifically bind to the capture agent-analyte complex, to produce a capture agent-analyte-detection agent complex linked to the magnetic particle; separating and washing the capture agent-analyte-detection agent complex; performing a quantitative measuring step on the oligonucleotide barcode sequences in the washed capture agent-analyte-detection agent complex in a multiplexed assay to detect and/or quantitate one or more of the analytes. In some embodiments, the quantitative measuring is performed by quantitative PCR (qPCR). In some embodiments, the method is performed on one to five analytes.

The present disclosure is also directed to a method of improving the specificity and/or reducing the background signal in a multiplex assay utilizing the systems and methods described herein.

In some embodiments, the analyte is a protein or biomarker, the biofluid is blood, plasma or serum, the capture agent is an antibody, and/or the detection agent is an antibody. In various embodiments, one or more of the method steps takes place in solution. In some embodiments, the method analysis multiple analytes in a sample. In some embodiments, the analytes are proteins. In some embodiments, one or more of the analytes is a soluble protein. In some embodiments, the proteins are separated from complex milieu comprising a plurality of various biomolecules, such as other proteins (including antibodies), DNA, lipids, etc.

The present disclosure also provides a method of detecting the presence of cancer in a subject. In some embodiments, the subject is not known to have cancer or has not undergone treatment for cancer. The present disclosure also provides a method of determining the prognosis of a subject with cancer. The present disclosure also provides a method of determining responsiveness to a cancer treatment in a subject. In various embodiments, the cancer is colorectal cancer, liver cancer, ovarian cancer, lung cancer, pancreatic cancer, stomach cancer, bladder cancer, or breast cancer.

In some embodiments, the results of the systems and/or methods disclosed herein are used as an input to generate a report. In embodiments, the report is in a paper or electronic format, and can include, for example, the results and quantitation of the analytes. In embodiments, the report can include, for example, information on the presence or absence of cancer, as determined by the methods or systems disclosed herein. Alternatively or additionally, in some embodiments the report contains information relating to genomic mutations, epigenetic rates of epigenetic features, or proteomic information. Embodiments of the methods or systems disclosed herein further include a step of communicating the report to a third party, such as the subject from whom the sample was derived, or a health care practitioner.

### BRIEF DESCRIPTION OF THE DRAWINGS

The accompanying drawings, which are incorporated in and constitute a part of this specification, illustrate certain embodiments, and together with the written description, serve to explain certain principles of the methods and systems disclosed herein. The description provided herein is better understood when read in conjunction with the accompanying drawings which are included by way of example and not by way of limitation. It will be understood that some or all of the figures may be schematic representations for purposes of illustration and do not necessarily depict the actual relative sizes or locations of the elements shown.

FIG. 1 is a schematic illustration of a complex used for multiplex analysis systems and methods according to an embodiment of the disclosure; the complex includes a magnetic particle, a capture agent, an analyte, a detection agent, and an oligonucleotide barcode.

FIG. 2 is an illustration of a workflow according to embodiments of the disclosure.

FIG. 3 is graph showing the results of qPCR detection of an analyte obtained by the multiplex analysis system and method according to an embodiment of the disclosure; concentration of analyte (IL-6) is on the X-axis (pg/ml) and delta cycle threshold values (Δ Ct) are on the Y-axis.

### DETAILED DESCRIPTION

Reference will now be made in detail to certain embodiments of the disclosure. While the disclosure will be described in conjunction with such embodiments, it will be understood that they are not intended to limit the disclosure to those embodiments. On the contrary, the disclosure is intended to cover all alternatives, modifications, and equivalents, which may be included within the disclosure as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids, reference to “a cell” includes a plurality of cells, and the like.

Numeric ranges are inclusive of the numbers defining the range. Measured and measurable values are understood to be approximate, taking into account significant digits and the error associated with the measurement. Also, the use of “comprise”, comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

The section headings used herein are for organizational purposes and are not to be construed as limiting the disclosed subject matter in any way. In the event that any document or other material incorporated by reference contradicts any explicit content of this specification, including definitions, this specification controls.

A “capture agent” as used herein refers to any molecule capable of capturing (directly or indirectly) an analyte (e.g., an analyte of interest) in a biological sample. Embodiments of a capture agent are capable of specifically binding an epitope of the analyte. Capture agents include, e.g., antibodies, nanobodies, aptamers, affimers, DARPins, and lectins.

A “detection agent” as used herein refers to any molecule capable of detecting (directly or indirectly) an analyte (e.g., an analyte of interest) that has been captured by the capturing agent. Embodiments of a detection agent are capable of specifically binding to an epitope of the analyte and/or to the capture agent-analyte complex.

As used herein, “capturing” one or more target molecule, such as one or more analyte, or one or more protein refers to preferentially isolating or separating the one or more target molecules from non-target molecules.

An “epitope” as used herein means the portion of a molecule or complex that is specifically bound by a binding molecule, such as a capture agent or a detection agent.

As used herein, the term “analyte” is to be construed broadly as any compound, molecule, or other substance of interest to be detected, identified, or characterized. An analyte also includes “auto-antibodies” that the subject has raised against an analyte, such as antibodies raised against cancer antigens.

“Specifically binds” in the context of a capturing agent or a detection agent means that under appropriate binding conditions, the capturing agent or detection agent binds to its target or analyte to form a stable complex, while at the same time formation of stable non-target complexes is minimized. For example, a capturing agent or detection agent binds to a target or analyte to a sufficiently greater extent.

The term “biological sample” or “sample” as used herein refers to any substance obtained from a subject. A sample may contain or be presumed to contain analytes for example those described herein (e.g., proteins, polypeptides, nucleic acids, carbohydrates, metabolites, etc.) from a subject. Examples of a biological sample (e.g., a bodily fluid) include whole blood, urine, saliva, cerebrospinal fluid, plasma, serum, ascites, sputum, sweat, tears, buccal sample, cavity rinse, or organ rinse.

As used herein, a “blood sample” refers to a sample containing whole blood or a component thereof (e.g., plasma, serum, buffy coat, plasma pellet).

The terms “polypeptide” or “protein” or “peptide” as used herein are intended to cover all naturally occurring proteins, as well as those which are recombinantly or synthetically produced. The terms also include naturally occurring modified forms of the proteins, such as glycosylated forms.

As used herein, a “barcode” is a nucleic acid sequence used to associate a detection agent, or a capture agent-analyte-detection agent complex, with the distinct analyte. In some embodiments, barcode sequences are of sufficient length or are sufficiently different from one another to allow the identification of the detection agent or capture agent-analyte-detection agent complex based on barcode sequences with which they are associated. In some embodiments, a barcode sequence may be contained within adapter sequences such that the barcode sequence is contained in the sequencing reads. In some embodiments, the barcode may comprise, or otherwise be referred to as, a unique molecular identifier (UMI).

As used herein, a “tag” is a molecule or sequence containing information that indicates a feature of the molecule to which the tag is associated. For example, molecules can bear a molecular tag or barcode (which distinguishes different molecules from one another (in both unique and non-unique tagging scenarios).

The term “target” as used herein refers to any analyte of interest that may be present in a sample. Example targets include, but are not limited to, biological macromolecules, peptides, proteins, or the like. The target may be one or more analyte molecules.

“Specifically binds” in the context of a capturing agent or a detection agent means that under appropriate binding conditions, the capturing agent or detection agent binds to its target or analyte to form a stable complex, while at the same time formation of stable non-target complexes is minimized. For example, a capturing agent or detection agent binds to a target or analyte to a sufficiently greater extent than to a nontarget, to ultimately enable capture or detection of the target or analyte.

An “antibody” as used herein broadly encompasses various antibody structures, including but not limited to monoclonal antibodies, polyclonal antibodies, multi-specific antibodies (e.g., bispecific antibodies), and antibody fragments so long as they exhibit the desired antigen-binding or epitope-binding activity.

An “antibody fragment” refers to a molecule other than an intact antibody that includes a portion of an intact antibody and that binds the antigen or epitope to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

It will be understood that when an element is referred to as being “attached” to, “connected” to, “coupled” with, etc., another element, it can be directly or indirectly attached to, connected to, coupled with the other element. Intervening elements may also be present.

“Or” is used in the inclusive sense, i.e., equivalent to “and/or,” unless the context requires otherwise.

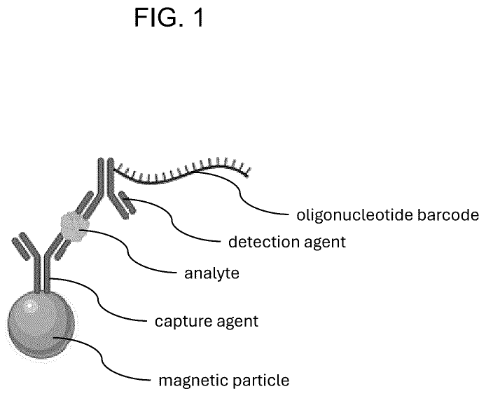

The present disclosure provides a multiplex analysis system and method for detecting and/or quantitating multiple analytes in a sample. Components of the system according to an embodiment are illustrated in FIG. 1.

According to various embodiments, the sample is contacted with an analyte binding agent or capturing agent and immobilized on a magnetic particle, followed by magnetic isolation of the particle and bound analyte. This complex is then contacted with a detection agent labeled with a nucleic acid detection probe specific to the analyte target. The combination of a plurality of analyte capture particles and cognate detection probes allows multiple analyte to be assayed simultaneously and in a multiplex manner.

For instance, embodiments of the systems and methods measure several cancer-specific protein biomarkers in a blood sample in a single assay. The systems and methods and their resulting measurements provide a resource for diagnosis of a disease or disease state, severity, or suitability for therapeutic intervention. The systems and methods described herein can be conducted in a decentralized laboratory space, where multiple samples can be processed simultaneously for cost efficiency or conducted in a near-patient setting.

According to various embodiments of the method, initial analyte capture from a sample is achieved using a capture agent bound or linked to a magnetic particle. A set of magnetic particles is provided, each magnetic particle in the set having a capture agent operatively linked to the particle. Each capture agent is configured to specifically bind to a distinct analyte. The set of magnetic particles is combined with the sample and each capture agent is allowed to specifically bind to the analyte, producing a complex containing the magnetic particle, capture agent and analyte. The capture agent-analyte complex linked to the magnetic particle is separated from the sample milieu by placing in a magnetic field, allowing for separation and washing of the particle complex from potentially interfering compounds.

According to other embodiments of the method, initial analyte capture from a sample is achieved using a capture agent that is not bound or linked to a magnetic particle, or prior to binding or linking the capture agent to the magnetic particles. In embodiments, a set of capture agents is provided, each capture agent being configured to specifically bind to a distinct analyte. The set of capture agents is combined with the sample in a solution and each capture agent is allowed to specifically bind to the analyte, forming a capture agent-analyte complex. The capture agent-analyte complex is subsequently operatively linked to a magnetic particle to form a capture agent-analyte complex linked to the magnetic particle. The capture agent-analyte complex linked to the magnetic particle is separated from the sample milieu by placing in a magnetic field, allowing for separation and washing of the particle complex from potentially interfering compounds.

According to additional embodiments of the method, initial analyte capture from a sample is achieved using a substrate having a plurality of capture agents operatively-linked thereto. In some embodiments, the substrate is one or more well of a plastic microplate. The substrate is provided having a plurality of capture agents operatively-linked to the substrate, each capture agent configured to specifically bind to a distinct analyte. The sample is combined with the substrate to allow each capture agent to specifically bind to the analyte, to form a capture agent-analyte complex on the substrate. The substrate and capture agent-analyte complex is washed to remove unbound compounds and potentially interfering compounds.

Solution-based approaches (e.g., wherein the capture agent is in solution at the time of combining, i.e., not bound to a solid support) may provide enhanced assay sensitivity as compared to array-based approaches (e.g., wherein the capture agent is bound to a magnetic particle or other solid substrate support). Solution-based approaches may provide more opportunities (as compared to array-based approaches) for a capture agent to interact with (e.g., bind) a target molecule because in such approaches the capture agent is free to diffuse throughout the sample. Accordingly, in some embodiments, the capture agent is in solution at the time of combining the sample with the capture agent. In such embodiments, the capture agent is not bound to a solid substrate, such as a bead or substrate support, at the time of combining the sample.

According to various embodiments, the detection and/or quantitation of each specific analyte in the washed capture agent-analyte complex is determined in a multiplex assay.

According to various embodiments, a set of detection agents is provided, and each detection agent in the set is configured to specifically bind to the capture agent-analyte complex linked to the magnetic particle, or the capture agent-analyte complex on the substrate. In addition, each detection agent has a specific oligonucleotide barcode sequence operatively linked to the detection agent.

In some embodiments, the washed capture agent-analyte complex linked to the magnetic particle is combined with the set of detection agents, and each detection agent is allowed to specifically bind to the capture agent-analyte complex to produce a capture agent-analyte-detection agent complex linked to the magnetic particle. This complex is separated from the mixture and excess reagent is removed by magnetic separation and washing.

In other embodiments, the washed capture agent-analyte complex on the substrate is combined with the set of detection agents, and each detection agent is allowed to specifically bind to the capture agent-analyte complex to produce a capture agent-analyte-detection agent complex on the substrate. The substrate and capture agent-analyte-detection agent is washed to remove excess reagent.

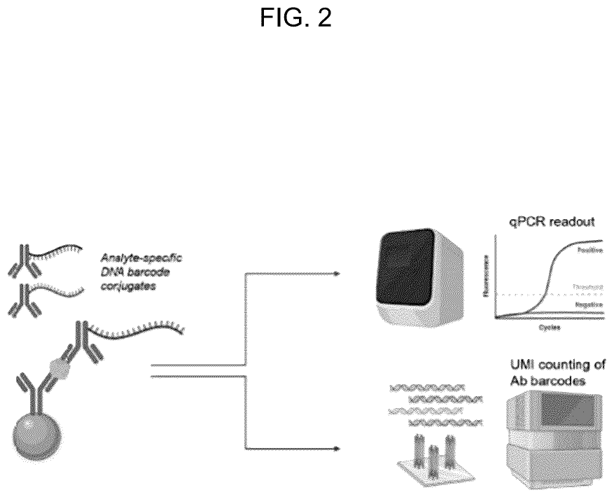

According to various embodiments, a quantitative measuring step is performed on the washed capture agent-analyte-detection agent complex, specifically on the oligonucleotide barcode sequence, to detect and/or quantitate each of the analytes. As illustrated in FIG. 2, embodiments of the method include performing multiplex qPCR to detect and/or quantitate the analytes. Other embodiments include performing next generation sequencing (NGS) to detect and/or quantitate the analytes.

In some embodiments, the methods utilize qPCR to analyze a plurality of analytes. The methods can be used in a multiplex assay to accurately detect and/or quantitate at least two analytes, for instance at least three, four, five, six, seven, eight, nine, or ten analytes. In some embodiments, the method is used in a quick diagnostic assay. As more analytes are included in the qPCR, the extent of spectral overlap may increase and can manifest as a reduction in detection sensitivity and increased background signal.

In some embodiments, the methods utilize NGS to analyze a plurality of analytes. In NGS, issues of overlap in dye absorbance and emission spectra can be obviated, as analyte quantitation is based upon digital counting of the specific barcode sequences. This provides very high sample throughput. According to various embodiments, the methods can be used in a multiplex assay to accurately detect and/or quantitate ten or more analytes, for instance 12, 15, 20, 25, 30, 40, 50, 60, 70, 80, 90, 100, 200, or more analytes.

According to various embodiments of the multiplex analysis system and method, a set of magnetic particles is provided, and each magnetic particle in the set has a capture agent operatively linked to the particle. In other embodiments, magnetic particles are provided without capture agents operatively linked to the particles. In various embodiments, the magnetic particle is a superparamagnetic particle that exhibits magnetic properties when placed in a magnetic field but retains no residual magnetism once removed from the magnetic field. In embodiments, the magnetic particles are magnetic beads, such as Dynabeads®. The terms “magnetic particles” and “magnetic beads” are used interchangeably herein.

According to various embodiments, the magnetic particles are coupled with a capture agent, which can be for example, an antibody, protein, aptamer, antigen, ligand, biomolecule, or any other molecule with an affinity for the desired target or analyte. In some embodiments, the capture agent is or includes an antibody or antibody fragment.

In some embodiments, the capture agent is covalently linked to the magnetic particle, or is linked to the magnetic particle by direct chemical conjugation. In some embodiments, the capture agent is indirectly attached to the magnetic particle, such as via streptavidin on the particle linking to a biotinylated capture agent, through oligonucleotide-mediated hybridization, or via secondary agent (e.g. secondary antibody) on the particle linking to the capture agent (e.g. primary antibody). In an embodiment, the capture agent is a biotinylated antibody linked to a streptavidin-coated Dynabead®. In the present disclosure, the term “operatively linked” means that the capture agent is coupled, covalently or non-covalently linked or bonded, or directly or indirectly attached to the magnetic particle by any of these techniques or by any other techniques known in the art.

According to various embodiments, the set of magnetic particles includes capture agents that are configured to specifically bind to distinct analytes. The set of magnetic particles coupled with a capture agent is combined with the sample and each capture agent in the set is allowed to bind to the specific analyte that may be present in the sample, producing a complex containing the magnetic particle, capture agent, and analyte. The set includes a plurality of distinct capture agents that together are directed to a plurality of distinct analytes. Thus, each capture agent is configured to specifically bind to one distinct analyte, but the set of magnetic particles includes distinct capture agents that in total are directed to a “multiplex” of analytes. In various embodiments, the capture agents in the set are configured to specifically bind to at least 1, 2, 3, 4, 5, 6, 7, 8, 9, 10, 12, 15, 20, 25, 30, 40, 50, 60, 70, 80, 90, 100, 200, or more distinct analytes.

In other embodiments of the method, magnetic particles are provided without capture agent coupled thereto. In the embodiments, one or more capture agent is combined with the sample in solution and each capture agent is allowed to bind to the specific analyte that may be present in the sample, producing a complex containing the capture agent and analyte. Subsequently, the capture agent-analyte complex is operatively-linked to the magnetic particles, producing a complex containing the magnetic particle, capture agent, and analyte. In embodiments, the capture agent-analyte linked to a magnetic particle complexes are pooled to produce a set of capture agent-analyte complexes.

According to various embodiments, while each of the capture agents in the set is configured to specifically bind to a distinct analyte, the set includes a plurality of distinct capture agents that together are directed to a plurality of distinct analytes. Thus, each capture agent is configured to specifically bind to one distinct analyte, but the set includes distinct capture agents that in total are directed to a “multiplex” of analytes. In various embodiments, the capture agents in the set are configured to specifically bind to at least 1, 2, 3, 4, 5, 6, 7, 8, 9, 10, 12, 15, 20, 25, 30, 40, 50, 60, 70, 80, 90, 100, 200, or more distinct analytes.

According to various embodiments, the formed capture agent-analyte complex linked to the magnetic particle (i.e., magnetic particle-capture agent-analyte complex) is separated from the sample milieu by placing in a magnetic field, allowing for separation and washing of the magnetic particle complex from potentially interfering compounds. Various embodiments of the method include separating and washing the magnetic particle complexes. Because the particles respond to a magnetic field, this allows the magnetic particle-capture agent-analyte complex to be rapidly and efficiently separated from the rest of the sample. The particle bound captured material in the complex is washed using a magnet or magnetic rack, the unbound or nonspecifically bound material is washed away from the captured material. In some embodiments, the washed particle-bound material (i.e., capture agent-analyte complex) is released from the complex in a suitable volume for further use. In some embodiments, the particle-bound material is used directly while still attached to the magnetic particle.

According to various embodiments, the formed capture agent-analyte-detection agent complex linked to the magnetic particle (i.e., magnetic particle-capture agent-analyte-detection agent complex) is separated from the reaction milieu by placing in a magnetic field, allowing for separation and washing of the magnetic particle complex from excess reagents. Various embodiments of the method include separating and washing the magnetic particle complexes. In some embodiments, the washed particle-bound material (i.e., capture agent-analyte-detection agent complex) is released from the complex in a suitable volume for further use. In some embodiments, the washed particle-bound material is used directly while still attached to the magnetic particle. In some embodiments, the oligonucleotide is released from the detection agent for further use, e.g., detection and/or quantitation.

According to various embodiments of the systems and methods, a quantitative measuring step is performed on the separated and washed capture agent-analyte-detection agent complex, linked or not linked to the magnetic particle, to detect and/or quantitate each of the analytes. In embodiments, quantitative measuring is performed on the oligonucleotide barcode sequence. Embodiments of the method include performing multiplex qPCR to detect and/or quantitate the analytes. Other embodiments include performing next generation sequencing (NGS) to detect and/or quantitate the analytes.

In various embodiments, the oligonucleotide is released from the complex prior to quantitative measuring. In some embodiments, the oligonucleotide is dissociated or released from the detection agent. In some embodiments, the oligonucleotide remains coupled to the capture agent and the capture agent-analyte-detection agent is dissociated from the magnetic particle, for example by disrupting the biotin-streptavidin interaction. In some embodiments, the oligonucleotide is not released and remains with the capture agent-analyte-detection agent complex, which remains coupled to the magnetic particle in the magnetic particle-capture agent-analyte-detection agent complex, prior to quantitative measuring.

According to various embodiments, the oligonucleotide contains a barcode sequence. A barcode can have, for example, between 10 and 100 nucleotides in length. In some embodiments, each barcode sequence includes at least 4, 6, 8, 10, 12, 14, 16, or more nucleotides in length. A collection of barcodes can have degenerate sequences or can have sequences having a certain hamming distance as desired for the specific purpose. In various embodiments, barcodes serve as a molecular tag through their individual sequences and also serve to identify and associate the detection agent (or a capture agent-analyte-detection agent complex) to the distinct analyte. In various embodiments, each barcode is unique and all or substantially all of the detection agents bear a different barcode tag.

According to various embodiments, the oligonucleotide barcode includes a unique molecular identifier (UMI) sequence. In some embodiments, the UMI is used to track and/or identify and associate the detection agent (or a capture agent-analyte-detection agent complex) to the distinct analyte. In some embodiments, the UMI can be used to reduce sequencing errors and PCR amplification bias.

According to various embodiments, the methods include performing multiplex qPCR to detect and/or quantitate the analytes that may or may not be in the sample. In various embodiments, qPCR is performed on the oligonucleotide barcode sequence. Embodiments of the method utilize qPCR to determine the number of oligonucleotide barcode tags and thus the original analyte concentration in the sample. In some embodiments, qPCR is preceded by one or more round of PCR amplification of the oligonucleotide prior to performing qPCR.

According to various embodiments, the methods include performing NGS to detect and/or quantitate the analytes that may or may not be in the sample. In various embodiments, NGS is performed on the oligonucleotide barcode sequence. Embodiments of the method utilize NGS to determine the number of oligonucleotide barcode tags and thus the original analyte concentration in the sample. In some embodiments, NGS is preceded by one or more round of PCR amplification of the oligonucleotide prior to performing NGS.

According to various embodiments, the oligonucleotides are sequenced using an Illumina sequencer. In various embodiments, the sequence reads generated by the sequencer are analyzed using bioinformatic tools and algorithms. The barcodes present in the sequenced oligonucleotides are used to identify the capture agent and their target analyte.

According to various embodiments, the quantitative measuring is performed using both qPCR and NGS, wherein a portion of the oligonucleotide barcode sequences is analyzed by qPCR and another portion of the oligonucleotide barcode sequences is analyzed by NGS.

According to various embodiments, the multiplex analysis method and system accurately quantitates one or more of the analytes in a linear dynamic range of at least three orders of magnitude, or at least four, five or six orders of magnitude. In some embodiments, the multiplex analysis method and system accurately quantitates each of the analytes in a linear dynamic range of at least three orders of magnitude, or at least four, five or six orders of magnitude.

According to various embodiments, the multiplex analysis method and system accurately quantitates one or more of the analytes to a level of at least 1 ng per mL of sample, or at least 0.5 ng/mL, 0.1 ng/mL, 500 pg/mL, 100 pg/mL, 50 pg/mL, 10 pg/mL, 5 pg/mL, 1 pg/mL, 0.5 pg/mL, 0.25 pg/mL, 0.1 pg/mL of sample, or lower.

According to various embodiments, the sample is a body fluid isolated from a subject, which includes for example, blood and fractions thereof, serum, plasma, urine, stool, saliva, sputum, cerebrospinal fluid, amniotic fluid, synovial fluid, pleural fluid, peritoneal fluid, and pericardial fluid. A sample can be in the form originally isolated from a subject or can have been subjected to further processing to remove or add components, or enrich for one or more component.

In some embodiments, the sample is obtained from a subject having a cancer or a precancer, an infection, transplant rejection, or other disease directly or indirectly affecting the immune system. In some embodiments, the sample is obtained from a subject suspected of having a cancer or a precancer, an infection, transplant rejection, or other disease directly or indirectly affecting the immune system. In some embodiments, the sample is obtained from a subject having a tumor. In some embodiments, the sample is obtained from a subject suspected of having a tumor. In some embodiments, the sample is obtained from a subject having neoplasia. In some embodiments, the sample is obtained from a subject suspected of having neoplasia. In some embodiments, the sample is obtained from a subject in remission from a tumor, cancer, or neoplasia (e.g., following chemotherapy, surgical resection, radiation, or a combination thereof). In any of the foregoing embodiments, the subject may be a human subject.

In some embodiments, the sample contains one or more analyte of interest for detection and/or quantitation, such as one or more biomarker. In some embodiments, the subject is not known to have cancer. In some embodiments, the subject has not previously undergone treatment for cancer. In some embodiments, the sample is obtained from a subject suspected of having neoplasia, a tumor, precancer, or cancer or previously diagnosed with neoplasia, a tumor, precancer, or cancer. In some embodiments, the subject has been treated with one or more cancer therapy, e.g., any one or more of chemotherapies, antibodies, vaccines or biologics.

According to various embodiments, the analyte of interest for detection and/or quantitation is one or more cancer-associated biomarker. A number of cancer-associated biomarkers are known and can be detected and quantitated according to embodiments of the present method and system. In various embodiments, the cancer-associated biomarker is a peptide or protein selected from, for example, oncofetal antigens (e.g., CEA, AFP), glycoprotein antigens or carbohydrate antigen (e.g. CA125, CA 19.9, CA 15-3), enzymes (e.g., PSA, ALP, NSE), hormone receptors (e.g., ER, PR), hormones (e.g., b-hCG, calcitonin), or other known biomolecules (e.g., VMA, 5HIAA, Mucins 1-24)).

In various embodiments, the analyte of interest is derived from tumor cells, cells in another disease state, or cells that altered due to the presence of a disease in the subject from which the cells are obtained. In some embodiments, one or more target proteins are derived from cell types not normally present in the type of bodily sample obtained from a subject.

In some embodiments, at least one analyte of interest is a glycoprotein carbohydrate. In some embodiments, one or more analyte is selected from RBI, TP53, PTEN, NF1, BRCA1, CEACAM1, CEACAM5, CEACAM6, EGFR, ErbB2, ErbB3, ErbB4, B-catenin, PD-L1, CTLA4, NYESO1, mesothelin, CA15-3, CA19-9, CA-125, CA27-29, and CA-72-4. In some embodiments, the one or more analyte is a protein known to show altered post-translational modification (e.g., altered glycosylation) in cancer. In some embodiments, one or more analyte is a cell type marker, such as an immune cell type marker or solid tissue cell type marker. In some embodiments, the solid tissue cell type marker is a marker present in colon, lung, breast, skin, prostate, stomach, pancreas, or liver cells.

In various embodiments, the analyte(s) of interest is a cancer associated peptide or protein marker are selected from 1p/19q deletion, HIAA, ACTH, AE1,3, ALK(D5F3), AFP, APC, ATRX, BOB-1, BCL-6, BCR-ABL1, beta-hCG, BF-1, BTAA, BRAF, GCDFP-15, BRCA1, BRCA2, b72.3, c-MET, calcitonin, CALR, calretinin, CA125, CA27.29, CA 19-9, CEA M, CEA P, CEA, CBFB-MYH11, CALA, c-Kit, syndical-1, CD14, CD15, CD19, CD2, CD20, CD200, CD23, CD3, CD30, CD33, CD4, CD45, CD5, CD56, CD57, CD68, CD7, CD79A, CD8, CDK4, CDK2, chromogranin A, creatine kinase isoenzymes, Cox-2, CXCL 13, cyclin D, CK 19, CYFRA 21-1, CK 20, CK5,6, CK 7, CAM 5.2, DCC, des-gamma-carboxy prothrombin, E-cadherin, EGFR T790M, EML4-ALK, ERBB2, ER, ESRI, FAP, gastrin, glucagon, HER-2/neu, SDHB, SDHC, SDHD, HMB45, HNPCC, HVA, beta-hCG, HE4, FBXW7, IDH1 R132H, IGH-CCND1, IGHV, IMP3, LOH, MUMI/IRF4, JAK exon 12, JAK2 V617F, Ki-67, KRAS, MCC, MDM2, MGMT, melan A, MET, metanephrines, MSI, MPL codon 515, Muc-1, Muckiest-4, MEN2, MYC, MYCN, MPO, myf4, myoglobin, myosin, napsin A, neurofilament, NSE P, NMP22, NPM1, NRAS, Oct 2, p16, p21, p53, pancreatic polypeptide, PTH, Pax-5, PAX8, PCA3, PD-L1 28-8, PIK3CA, PTEN, ERCC-1, Ezrin, STK11, PLAP, PML/RARa translocation, PR, proinsulin, prolactin, PSA, PAP, PGP, RAS, ROS1, S-100, S100A2, S100B, SDHB, serotonin, SAMD4, MESOMARK, squamous cell carcinoma antigen, SS18 SYT 18q11, synaptophysin, TIA-1, TdT, thyroglobulin, TNIK, TP53, TTF-1, TNF-alpha, TRAFF2, urovysion, VEGF, or combinations thereof.

In some embodiments, the analyte of interest may be selected form an interleukin protein family, including IL-1, IL-2, IL-6, IL10, IL-12, and IL-17 families.

In some embodiments, the analyte of interest may include a receptor for advanced glycation endproducts (RAGE).

In various examples, the cancer-associated biomarker is associated one or more of colorectal cancer, liver cancer, ovarian cancer, lung cancer, pancreatic cancer, stomach cancer, bladder cancer, or breast cancer.

In some embodiments, at least one analyte of interest is associated with a disease, two or more of a plurality of analytes are associated with a disease, or each of a plurality of analytes is associated with a disease, e.g., cancer. In some embodiments, at least one analyte is a protein that is differentially post-translationally modified in tumor cells relative to healthy cells of the same tissue type. In some embodiments, at least one analyte is upregulated in tumor cells relative to healthy cells of the same tissue type.

In some embodiments, at least one analyte of interest, two or more of a plurality of analytes, or each of a plurality of analyte is a cell type marker. In some embodiments, the cell type markers are selected from markers for immune cells and solid tissue cells. In some embodiments, the cell type markers are selected from markers for colon, lung, breast, ovarian, skin, prostate, stomach, pancreas, and liver cell type markers.

In some embodiments, the detecting and/or quantitating step facilitates disease diagnosis or identification of appropriate treatments. In some embodiments, the presence of or a change in the levels of one or more analytes is indicative of the presence of a disease or disorder in a subject, such as cancer, precancer, an infection, transplant rejection, or other disorder that causes changes in cell death.

Various embodiments of the present disclosure can be used to diagnose presence of conditions, such as cancer or precancer, in a subject, to characterize conditions (e.g., staging cancer or determining heterogeneity of a cancer), monitor response to treatment of a condition, effect prognosis risk of developing a condition or subsequent course of a condition. Various embodiments of the present disclosure can also be useful in determining the efficacy of a particular treatment option. Successful treatment options may increase the amount of target analyte detected in a subject's blood if the treatment is successful as the cancer dies and sheds DNA and proteins, among other things. In other examples, this may not occur. Additionally, if a cancer is observed to be in remission after treatment, embodiments of the present methods can be used to monitor residual disease or recurrence of disease.

In various embodiments, the subject is determined to have cancer. In some embodiments, the present method is used to determine a cancer type, such as colorectal cancer, liver cancer, ovarian cancer, lung cancer, pancreatic cancer, stomach cancer, bladder cancer, or breast cancer, based on the analyte detected.

In some embodiments, the present methods are used for screening for a cancer, or in a method for screening cancer. For example, the sample can be a sample from a subject who has not been previously diagnosed with cancer. In some embodiments, the subject may or may not have cancer. In some embodiments, the subject may or may not have an early-stage cancer. In some embodiments, the subject has one or more risk factor for cancer, such as tobacco use (e.g., smoking), being overweight or obese, having a high body mass index (BMI), being of advanced age, poor nutrition, high alcohol consumption, or a family history of cancer.

In some embodiments, the methods and systems disclosed herein are used to identify customized or targeted therapies to treat a given disease or condition in patients based on the presence of one or more analytes of interest.

In some embodiments, the methods and systems disclosed herein are used for determining a risk of cancer recurrence in a subject. In some embodiments, the methods and systems are used for classifying a subject as being a candidate for a subsequent cancer treatment.

In some embodiments, the present methods and systems are used to monitor one or more aspects of a condition in a subject over time, such as a subject's response to receiving a treatment for a condition (such as a response to a chemotherapeutic or immunotherapeutic), the severity of the condition (such as a cancer stage) in the subject, a recurrence of the condition (such as a cancer), or the subject's risk of developing the condition (such as a cancer) and/or to monitor a subject's health as part of a preventative health monitoring program (such as to determine whether or when a subject is in need of further diagnostic screening). In some embodiments, the monitoring includes analysis of at least two samples collected from a subject at at least two different time points as described herein.

In some embodiments, the methods and systems disclosed herein relate to identifying and administering therapies, such as customized therapies, to patients or subjects. In some embodiments, determination of the levels of particular target analytes facilitates selection of appropriate treatment.

In some embodiments, a sample is isolated or obtained from a subject and transported to a site of sample analysis. In embodiments, the sample is preserved and shipped at a desirable temperature, e.g., room temperature, 4° C., −20° C., and/or −80° C. In other embodiments, a sample is isolated or obtained from a subject at the site of the sample analysis. The subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines, or biologics. The subject may be in remission. The subject may or may not be diagnosed of being susceptible to cancer or have any cancer-associated genetic mutations/disorders.

According to various embodiments, different sets of capture agents, either operatively-linked or not to the magnetic particles, can be utilized in embodiments of the method. Different sets of capture agents can be mixed together to form bead libraries or assay panels that can detect and/or quantify multiple analytes in a single sample.

In some embodiments, the particles are additionally modified to include moieties to reduce the effects of non-specific binding of plasma/serum components, such as through the incorporation of additional low-binding polymeric moieties, including polyethylene glycol, dextrans and zwitterionic compounds.

According to various embodiments, the methods and systems of the present disclosure are used in combination with other methods and systems that analyze other analytes, for example cell-free DNA molecules, cell-free RNA molecules, antibodies, and metabolites. For example, embodiments of the methods and systems can be combined with one or more of whole-genome sequencing (WGS), whole genome bisulfite sequencing (WGSB), methylation analysis, small-RNA sequencing, enzyme-linked immunosorbent assay (ELISA), proximity extension assay (PEA), protein microarray, and mass spectrometry.

According to various embodiments, the methods and systems of the present disclosure can be used in combination with other methods and systems that analyze, for example, genomic biomarkers based on the analysis of DNA profiles, sequences or modifications; transcriptomic biomarkers based on the analysis of RNA expression profiles, sequences or modifications; proteomic or protein biomarkers based on the analysis of protein profiles, sequences or modifications; and metabolomic biomarkers based on the analysis of metabolites abundance.

According to various embodiments, methods of the present disclosure are implemented using, or with the aid of, a computer system programmed or otherwise configured to implement the methods of the present disclosure. In various embodiments, the computer system regulates various aspects of sample preparation, sequencing, and/or analysis. In some embodiments, the computer system is configured to perform sample preparation and sample analysis, including (where applicable) nucleic acid sequencing, e.g., according to any of the methods disclosed herein.

According to various embodiments, the methods and systems of the present disclosure incorporate the use of machine learning to analyze multiple analytes from a biological sample. In embodiments, machine learning enables large-scale statistical approaches and automated characterization of signal strength.

Further provided by the present disclosure are kits that include the compositions as described herein. In various embodiments, the kits are for use in performing the methods as described herein. In some embodiments, the kit includes a set of magnetic particles, each magnetic particle having a capture agent operatively-linked to the particle, each capture agent configured to specifically bind to a distinct analyte and form a capture agent-analyte complex. In some embodiments, the kit includes a set of detection agents, each detection agent in the set configured to specifically bind to the capture agent-analyte complex, and each detection agent having a specific oligonucleotide barcode sequence operatively linked to the detection agent. In some embodiments, the kit includes reagents for detecting and/or quantitating the presence or level of target analytes.

In various embodiments, the kit includes a set of capture agents, each capture agent configured to specifically bind to a distinct analyte and form a capture agent-analyte complex. In various embodiments, the kit includes magnetic particles configured to operatively-link with the set of capture agents.

In various embodiments, the kit includes a substrate having a plurality of capture agents operatively-linked to the substrate, each capture agent in the plurality configured to specifically bind to a distinct analyte. In some embodiments, the substrate is one or more well of a plastic microplate. In some embodiments, the one or more well is randomly coated with a plurality of capture agents. In other embodiments, the one or more well is coated with a plurality of capture agents through specific spatial localization.

In some embodiments, the kit includes additional elements disclosed herein. In some embodiments, the kit includes instructions for performing a method disclosed herein.

While embodiments of the present disclosure have been shown and described herein, it will be clear to those skilled in the art that such embodiments are provided by way of example only. It is not intended that the disclosure be limited by the specific examples provided within the specification. While the disclosure has been described, the descriptions and illustrations of the embodiments herein are not meant to be construed in a limiting sense. Numerous variations, changes, and substitutions will now occur to those skilled in the art without departing from the true scope of the disclosure.

Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations, or equivalents.

While inventive concepts have been described and illustrated herein by reference to certain embodiments, various changes and further modifications may be made by those of ordinary skill in the art without departing from the spirit of the inventive concept, the scope of which is to be determined by the following claims and their equivalents to be covered thereby.

### EXAMPLES

The following examples are provided to illustrate certain aspects of the disclosed methods. The examples do not limit the disclosure or embodiments.

### Example 1: Detection of IL-6

Magnetic particle-capture agent. Anti-human IL-6 (MQ2-39C3, BioLegend) was conjugated to NHS-PEG4-Biotin at a molar ratio of 25:1 for 2 hr at room temperature and purified by gel filtration using a Bio-Rad P-30 desalting media. The biotinylated antibody was linked to streptavidin-coated M-270 DynaBeads at a ratio of 10 ug antibody per mg bead in PBS buffer for 30 min at room temperature. The beads were washed 5 times and resuspended in assay buffer (PBS, 0.1% BSA, 0.05% Tween-20, 0.05% sodium azide) at a final concentration of approximately 0.5 mg/mL particle concentration.

Detection agent-oligonucleotide barcode. Oligonucleotide conjugated detection antibody was prepared by activation of anti-human IL-6 with 25 equivalents of trans cyclooctene-PEG4-TFP ester, followed by purification using Bio-Rad P-30 desalting media. 5′ amine-terminated detection oligonucleotide (/5AmMC12/GATCTACACTCTTTCCCTACACGACGCTCTTCCGATCTCGATGTGTCGGA AGAGCACACGTCTGAACTCCAGTCAC) was reacted with 25 equivalents of methyl tetrazine-PEG4-TFP ester in PBS for 2 hrs, followed by purification using Bio-Rad P-30 desalting media. The activated oligonucleotide was mixed with activated antibody at a molar ratio of 4:1, allowed to react for 12 hrs at RT and stored at 2-8° C.

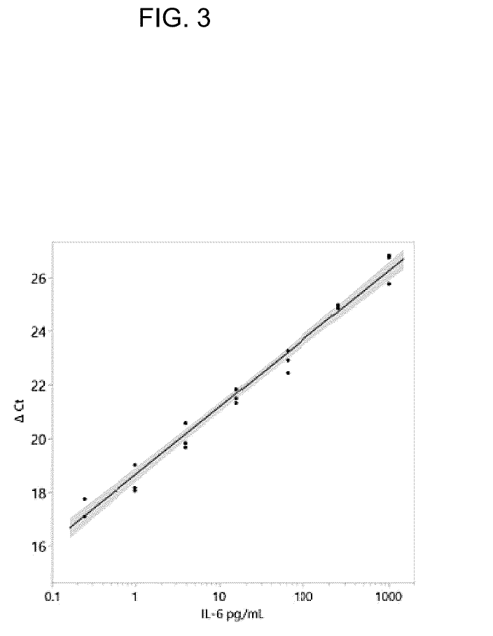

IL-6 sample quantitation. Recombinant human IL-6 was serially diluted in assay buffer and 40 uL aliquots incubated with 2.5 ug of capture particles for 1 hr at room temperature in a Lo-bind twin-tec 96 well plate (Eppendorf). The plates were subjected to magnetic separation and washing in assay buffer. The purified particles were contacted with 40 uL of 0.1 ug/mL detection antibody-oligo conjugate for 1 hr at room temperature, followed by magnetic separation and washing and final resuspension in 50 uL of assay buffer. 20 uL of the solution was transferred to a secondary qPCR-compatible microplate containing 20 uL of 2X concentrate qPCR SYBR green mastermix, followed by qPCR (QuantStudio Flex 6, Applied Biosystems, 10 min 95° C. 10 min, 40 cycles 60° C. 45s, 95° C. 15s). Cycle threshold (Ct) values were obtained for the different IL-6 dilution points from the instrument software output (QuantStudio), and the results are shown in FIG. 3. A linear dynamic range over 4-5 orders of magnitude can be readily achieved with a limit of detection approx. 0.25 pg/mL IL-6.

What is claimed is:

1. A multiplex analysis method for detecting and/or quantitating multiple analytes in a sample, the method comprising:
providing a set of magnetic particles, each magnetic particle in the set comprising a capture agent operatively-linked to the particle, each capture agent configured to specifically bind to a distinct analyte;
combining the sample and set of magnetic particles to allow each capture agent to specifically bind to the analyte, to produce a capture agent-analyte complex linked to the magnetic particle;
separating and washing the capture agent-analyte complex;
providing a set of detection agents, each detection agent in the set configured to specifically bind to the capture agent-analyte complex, and each detection agent comprising a specific oligonucleotide barcode sequence operatively linked to the detection agent;
combining the washed capture agent-analyte complex and the set of detection agents to allow the detection agent to specifically bind to the capture agent-analyte complex, to produce a capture agent-analyte-detection agent complex linked to the magnetic particle;
separating and washing the capture agent-analyte-detection agent complex;
performing a quantitative measuring step on the oligonucleotide barcode sequences in the washed capture agent-analyte-detection agent complex in a multiplexed assay to detect and/or quantitate each of the analytes.

2. The method of claim 1, wherein the quantitative measuring is performed by quantitative PCR (qPCR) or by next generation sequencing (NGS).

3. The method of claim 2, wherein the quantitative measuring further comprises one or more round of PCR amplification prior to performing the qPCR or NGS.

4. The method of any one of claims 1-3, wherein the oligonucleotide barcode comprises a unique molecular index (UMI) sequence.

5. The method of any one of claims 1-4, wherein the multiplex analysis detects and/or quantitates forty or more analytes.

6. The method of any one of claims 1-5, wherein an analyte is quantitated in a linear dynamic range of at least about four orders of magnitude.

7. The method of any one of claims 1-6, wherein the sample is blood, plasma, or serum.

8. The method of any one of claims 1-7, wherein the analytes are cancer biomarkers.

9. The method of any one of claims 1-8, wherein the capture agent is an antibody.

10. The method of any one of claims 1-9, wherein the capture agent is linked to the magnetic particle by direct chemical conjugation, streptavidin-biotin binding, or oligonucleotide-mediated hybridization.

11. The method of any one of claims 1-10, wherein the oligonucleotide barcode sequence is linked to the detection agent by direct chemical conjugation, streptavidin-biotin binding, or oligonucleotide-mediated hybridization.

12. The method of any one of claims 1-11, wherein the capture agent is biotinylated and is linked to a streptavidin-coated magnetic particle, and the oligonucleotide barcode sequence is linked to the detection agent by direct chemical conjugation.

13. The method of any one of claims 1-12, having a detection level of at least 0.25 pg analyte per mL of sample.

14. A multiplex analysis method for detecting and/or quantitating multiple analytes in a sample, the method comprising:
providing a set of capture agents, each capture agent in the set configured to specifically bind to a distinct analyte;
combining the set of capture agents and the sample in a solution, to allow each capture agent to specifically bind to the analyte and form a capture agent-analyte complex;
operatively linking the capture agent-analyte complex to magnetic particles to form a capture agent-analyte complex linked to the magnetic particle;
separating and washing the capture agent-analyte complex;
providing a set of detection agents, each detection agent in the set configured to specifically bind to the capture agent-analyte complex, and each detection agent comprising a specific oligonucleotide barcode sequence operatively linked to the detection agent;
combining the washed capture agent-analyte complex and the set of detection agents to allow the detection agent to specifically bind to the capture agent-analyte complex, to produce a capture agent-analyte-detection agent complex linked to the magnetic particle;
separating and washing the capture agent-analyte-detection agent complex;
performing a quantitative measuring step on the oligonucleotide barcode sequences in the washed capture agent-analyte-detection agent complex in a multiplexed assay to detect and/or quantitate each of the analytes.

15. The method of claim 14, wherein the quantitative measuring is performed by quantitative PCR (qPCR) or by next generation sequencing (NGS).

16. The method of claim 15, wherein the quantitative measuring further comprises a round of PCR amplification prior to performing the qPCR or NGS.

17. The method of any one of claims 14-16, wherein the oligonucleotide barcode comprises a unique molecular index (UMI) sequence.

18. The method of any one of claims 14-17, wherein the multiplex analysis detects and/or quantitates forty or more analytes.

19. The method of any one of claims 14-18, wherein an analyte is quantitated in a linear dynamic range of at least about four orders of magnitude.

20. The method of any one of claims 14-19, wherein the sample is blood, plasma, or serum.

21. The method of any one of claims 14-20, wherein the analytes are cancer biomarkers.

22. The method of any one of claims 14-21, wherein the capture agent is an antibody.

23. The method of any one of claims 14-22, wherein the capture agent-analyte complex is linked to the magnetic particle by direct chemical conjugation, streptavidin-biotin binding, or oligonucleotide-mediated hybridization.

24. The method of any one of claims 14-23, wherein the oligonucleotide barcode sequence is linked to the detection agent by direct chemical conjugation, streptavidin-biotin binding, or oligonucleotide-mediated hybridization.

25. The method of any one of claims 14-24, wherein the capture agent is biotinylated and is linked to a streptavidin-coated magnetic particle, and the oligonucleotide barcode sequence is linked to the detection agent by direct chemical conjugation.

26. The method of any one of claims 14-25, having a detection limit of at least 0.25 pg analyte per mg of sample.

27. A kit comprising:
a set of magnetic particles, each magnetic particle in the set comprising a capture agent operatively-linked to the particle, each capture agent configured to specifically bind to a distinct analyte and form a capture agent-analyte complex; and
a set of detection agents, each detection agent in the set configured to specifically bind to the capture agent-analyte complex, and each detection agent comprising a specific oligonucleotide barcode sequence operatively linked to the detection agent.

28. A kit comprising
a set of capture agents, each capture agent configured to specifically bind to a distinct analyte and form a capture agent-analyte complex; and
a set of detection agents, each detection agent in the set configured to specifically bind to the capture agent-analyte complex, and each detection agent comprising a specific oligonucleotide barcode sequence operatively linked to the detection agent.

29. A multiplex analysis method for detecting and/or quantitating multiple analytes in a sample, the method comprising:
providing a substrate comprising a plurality of capture agents operatively-linked to the substrate, each capture agent in the plurality configured to specifically bind to a distinct analyte;
combining the sample with the substrate to allow each capture agent to specifically bind to the analyte, to produce a capture agent-analyte complex on the substrate;
washing the substrate and capture agent-analyte complex;
providing a set of detection agents, each detection agent in the set configured to specifically bind to a distinct capture agent-analyte complex, and each detection agent comprising a specific oligonucleotide barcode sequence operatively linked to the detection agent;
combining the washed capture agent-analyte complex with the set of detection agents to allow the detection agent to specifically bind to the capture agent-analyte complex, to produce a capture agent-analyte-detection agent complex on the substrate;
washing the substrate and capture agent-analyte-detection agent complex;
performing a quantitative measuring step on the oligonucleotide barcode sequences in a multiplexed assay to detect and/or quantitate each of the analytes.

30. The method of claim 29, wherein the substrate is one or more well of a plastic microplate.

31. The method of claim 30, wherein the one or more well is randomly coated with the plurality of capture agents.

32. The method of claim 30, wherein the one or more well is coated with the plurality of capture agents through specific spatial localization.

33. A method for detecting and/or quantitating multiple analytes in a sample from a subject, the method comprising:
providing a set of magnetic particles, each magnetic particle in the set comprising a capture agent operatively-linked to the particle, each capture agent configured to specifically bind to a distinct analyte;
combining the sample and set of magnetic particles to allow each capture agent to specifically bind to the analyte, to produce a capture agent-analyte complex linked to the magnetic particle;
separating and washing the capture agent-analyte complex;
providing a set of detection agents, each detection agent in the set configured to specifically bind to the capture agent-analyte complex, and each detection agent comprising a specific oligonucleotide barcode sequence operatively linked to the detection agent;
combining the washed capture agent-analyte complex and the set of detection agents to allow the detection agent to specifically bind to the capture agent-analyte complex, to produce a capture agent-analyte-detection agent complex linked to the magnetic particle;
separating and washing the capture agent-analyte-detection agent complex;
performing a quantitative measuring step on the oligonucleotide barcode sequences in the washed capture agent-analyte-detection agent complex in a multiplexed assay to detect and/or quantitate each of the analytes.

34. The method of claim 33, wherein the quantitative measuring step comprises determining the presence of a cancer in the subject based on the detection of one or more analytes in the sample that are associated with the cancer.
